(19) United States
(12) Patent Application Publication
(10) Pub. No.: US 2013/0302247 A1
(43) Pub. Date: Nov. 14, 2013

(54) SYNTHESIS OF FLUOROARACHIDONIC ACID AND METHODS OF USE THEREOF

(71) Applicants: The United States of America, as represented by the Secretary, Department of Health and Human Serv, Bethesda, MD (US); The Regents of the University of California, Oakland, CA (US)

(72) Inventors: Ramaiah Pichika, San Clemente, CA (US); William C. Eckelman, Bethesda, MD (US); Stanley I. Rapoport, Washington, DC (US); Ameer Taha, Washington, DC (US); Kishore Kotta, San Diego, CA (US)

(21) Appl. No.: 13/890,884

(22) Filed: May 9, 2013

Related U.S. Application Data
(60) Provisional application No. 61/644,970, filed on May 9, 2012.

(51) Int. Cl. A61K 51/04

(52) U.S. Cl. CPC A61K 51/0402
USPC 424/1.89; 554/88; 554/150; 554/175

(57) ABSTRACT

There are provided, inter alia, methods for diagnosis of the extent of neuroinflammation in a subject. The methods include administering an ¹⁸F-labeled arachidonic acid to a subject in need thereof, obtaining a positron emission tomography (PET) scan of the subject, and determine the extent of neuroinflammation from the PET scan. There are provided, inter alia, methods of synthesis of reagents useful for the production of an ¹⁸F-labeled arachidonic acid.

### CROSS-REFERENCE TO RELATED APPLICATION

This patent application claims the benefit of U.S. Provisional Patent Application No. 61/644,970, filed May 9, 2012, which is incorporated by reference herein in its entirety.

### BACKGROUND OF THE INVENTION

Arachidonic acid (AA, 20:4n-6) is a polyunsaturated fatty acid found in high concentratio in brain phospholipids, and is preferentially incorporated into phosphatidylinositol (PtdIns) and choline glycerophospholipids (ChoGpls) (1, 2). AA release from the stereoselective sn-2 numbered position of brain phospholipids is mediated by AA-selective calcium-dependent cytosolic phospholipase A₂ (group IV), which is coupled to G-protein receptors (serotonergic 5-HT2A/2C, cholinergic muscarinic M1-3, dopaminergic D2) or ionotropic N-methyl-D-aspartate (NMDA) neurotransmission (3). AA metabolism is upregulated in neuroinflammation, excitotoxicity and other brain disturbances, including Alzheimer's disease, bipolar disorder, stroke, and HIV-1 associated dementia (4-8).

Regional disturbances in brain AA metabolism have been imaged with [1-¹⁴C]AA in animals using ex vivo autoradiography, which show upregulated AA metabolism during bacterial lipopolysaccharide or NMDA-induced neuroinflammation and excitotoxicity (9, 10), G-protein neuroreceptor signaling (3, 11) and in an HIV transgenic rat model of neuroinflammation (4, 12). In humans, brain AA metabolism has been imaged with positron emitting tomography (PET), using [¹¹C]AA as a radiotracer, at rest and during functional activation (6, 13, 14). Measurements have been shown to be independent of changes in cerebral blood flow, thus markers of metabolism (13, 15). Upregulated [¹¹C]AA metabolism associated with neuroinflammation has been demonstrated in Alzheimer's disease patients with PET, particularly in regions reported to have high densities of senile plaques and activated microglia (6), suggesting the utility of [¹¹C]AA for imaging regional disturbances in brain AA metabolism in neurodegenerative disorders with a neuroinflammatory component.

The routine use of [¹¹C]AA as a biotracer currently is limited to a few centers able to synthesize the tracer on-site and to administer it directly to patients, due to its short radioactive half-life (20.4 min). Also, challenges with the synthetic chemistry caused by using an unstable bromo fatty acid precursor (2) in a Grignard reaction and the inability to capture high-resolution images due to the short half-life of the [¹¹C]fatty acid tracer, have limited the exploitation of this tracer as an in vivo biomarker of disturbed AA metabolism (16, 17). Thus, gaining wider clinical use of [¹¹C]AA as a biomarker for neuroinflammation, particularly in clinical centers and hospitals that cannot synthesize it, would require the development of a stable analogue with a longer radioactive half-life.

The use of radiofluorinated tracers in lieu of ¹¹C-labeled tracers has provided a breakthrough in the field of PET imaging of regional brain metabolism in normal and pathological conditions including neuroblastoma, infection and tumor, due to the longer radioactive half-life of [¹⁸F]fluorinated compounds (109.8 min) relative to [¹¹C]tracers (20.4 min) (18-22). Fluoro-2-deoxyglucose for [¹¹C]glucose and 3′-fluoro-thymidine (FLT) for [¹¹C]thymidine are prime examples (23). Following the trend to develop a clinically useful imaging marker with fluorinated analogues for [¹¹C], fluorinated AA ([¹⁸F]FAA) was developed by other investigators (24). But, it is difficult to synthesize high yields of the fatty acid tracer with the published synthetic method (25). Also, incorporation and rates of incorporation of [¹⁸F]FAA into specific brain phospholipids was not confirmed in vivo, although its presence in brain total lipid and aqueous extracts was reported in mice (25). The phospholipid kinetics and distribution are key to using FAA as a tracer for AA.

In the present study, we developed a high-yield synthetic method for non-radioactive, fluorinated [¹⁹F]FAA (FAA), and applied our in vivo kinetic infusion model (2, 26) to quantify its incorporation and rates of incorporation in brain phospholipids of unanesthetized mice using gas-chromatography mass spectrometry (GC/MS). Without wishing to be bound by any theory, it is believed that GC/MS and [¹⁹F]FAA can be used for these studies given that we can identify and quantitate the mass of the FAA in each phospholipid in the brain with high sensitivity, whereas using [¹⁸F]FAA we would have minimal count rates after the extraction and would need to rely on GC retention times with standard reference compounds to identify the FAA. The use of GC/MS and [¹⁹F]FAA is a necessary but not sufficient step, but it has the potential to validate FAA as a true tracer of AA.

Based on the reported distribution of [1-¹⁴C]AA in brain phospholipids in mice (2), we hypothesized that the fluorinated arachidonic acid would be preferentially incorporated into brain ChoGpls and PtdIns after a 5-minute intravenous infusion. [1-¹⁴C]AA does not differ chemically from the natural compound, thus its incorporation is a direct measure of incorporation and distribution of naturally occurring unesterified AA. Our results confirmed the preferential incorporation of [¹⁹F]FAA into ChoGpls and PtdIns, which was similar to the reported incorporation pattern of [¹⁴C]AA in mice, and at equivalent incorporation rates (2).

### BRIEF SUMMARY OF THE INVENTION

In a first aspect, there is provided a method of diagnosis of the extent of neuroinflammation in a subject. The method includes administering an ¹⁸F-labeled arachidonic acid to a subject in need thereof. The method further includes obtaining a positron emission tomography (PET) scan of the subject. The method further includes determining the extent of neuroinflammation of said subject from said PET scan. The term “neuroinflammation” means, in the customary sense, inflammation of neural tissue including the brain and other components of the neural system of a subject, as known in the art.

In another aspect, there is provided a method for producing methyl 20-(p-toluenesulfonyloxy)-5,8,11,14-eicosatetraenoate, which method includes reacting methyl 20-hydroxy-5,8,11,14-eicosatetraenoate with tosyl chloride and triethylamine in dichloromethane.

In another aspect, there is provided a method for producing methyl 20-fluoroarachidonate, which method includes reacting methyl 20-(p-toluenesulfonyloxy)-5,8,11,14-eicosatetraenoate with tetra-n-butylammonium fluoride in tethydrofuran.

In another aspect, there is provided a method for separation of geometric isomers of methyl 20-hydroxy-5,8,11,14-eicosatetraenoate, which method includes subjecting a mixture of E and Z isomers of methyl 20-hydroxy-5,8,11,14-eicosatetraenoate to flash column chromatography. Methods and devices for flash column chromatography are known in the art.

### BRIEF DESCRIPTION OF THE DRAWINGS

FIG. 1 depicts the time course of plasma FAA concentration during intravenous infusion of 0.3 or 1.6 nmol/FAA over 5 minutes. Legend: 0.3 nmol FAA (diamonds); 1.6 nmol FAA (boxes). Values are mean±SD (standard deviation) of n=2 (low cost) or n=3 (high dose).

FIG. 2A depicts FAA concentration in brain lipids (nmol/g wet weight) following 5-min infusion with low (0.3 nmol) or high dose (1.6 nmol). Legend: 0.3 nmol FAA (closed); 1.6 nmol FAA (open). Abbreviations: TL, total lipids; PL, phospholipids; ChoGpl, choline glycerophospholipid; PtdIns, phosphatidylinositol; EtnGpl, ethanolamine glycerophospholipid; PtdSer, phosphatidylserine. Values are mean±SD of n=3 mice for the low dose, except for EtnGlp and PtdSer, for which n=2; n=2 for high dose. FIG. 2B depicts percent distribution of FAA in brain phospholipid subfractions. Bars represent mean±SD. Bars with different superscripts are significantly different from each other by one-way ANOVA followed by Tukey's post-doc test. n=5 for ChoGlp and PtdIns; n=4 for EtnGlp and PtdSer.

FIG. 3 depicts FAA incorporation coefficient, k*, in brain lipids following 5-min infusion with low (0.3 nmol) or high dose (1.6 nmol). Abbreviations: as in FIG. 2. Values are mean±SD of n=3 mice for the low dose, except for EtnGlp and PtdSer, for which n=2.

### DETAILED DESCRIPTION OF THE INVENTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched chain, or combination thereof, which may be fully saturated, mono- or polyunsaturated and can include di- and multivalent radicals, having the number of carbon atoms designated (i.e., C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—).

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, consisting of at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any interior position of the heteroalkyl group or at the position at which the alkyl group is attached to the remainder of the molecule. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two heteroatoms may be consecutive, such as, for example, —CH₂—NH—OCH₃.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl, and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

The term “oxo,” as used herein, means an oxygen that is double bonded to a carbon atom.

The term “alkylsulfonyl,” as used herein, means a moiety having the formula —S(O₂)—R, where R′ is an alkyl group as defined above. R′ may have a specified number of carbons (e.g., “C₁-C₄ alkylsulfonyl”).

Each of the above terms (e.g., “alkyl,” “heteroalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN, and —NO₂ in a number ranging from zero to (2 m′+1), where m′ is the total number of carbon atoms in such radical. R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN, —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) —OH, —NH₂, —SH, —CN, —CF₃, —NO₂, oxo, halogen, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, and heteroaryl, substituted with at least one substituent selected from:
   (i) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and
   (ii) alkyl, heteroalkyl, cycloalkyl, heterocycloalkyl, aryl, and heteroaryl, substituted with at least one substituent selected from:
      (a) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and
      (b) alkyl, heteroalkyl, cycloalkyl, heterocycloalkyl, aryl, or heteroaryl, substituted with at least one substituent selected from: oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, and unsubstituted heteroaryl.

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₄-C₈ cycloalkyl, and each substituted or unsubstituted heterocycloalkyl is a substituted or unsubstituted 4 to 8 membered heterocycloalkyl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₅-C₇ cycloalkyl, and each substituted or unsubstituted heterocycloalkyl is a substituted or unsubstituted 5 to 7 membered heterocycloalkyl.

The term “pharmaceutically acceptable salts” is meant to include salts of the active compounds that are prepared with relatively nontoxic acids or bases, depending on the particular substituents found on the compounds described herein. When compounds described herein contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds described herein contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydroiodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds described herein contain both basic and acidic functionalities that allow the compounds to be converted into either base or acid addition salts.

Thus, the compounds described herein may exist as salts, such as with pharmaceutically acceptable acids. In some embodiments, the compounds and compositions described herein includes such salts. Examples of such salts include hydrochlorides, hydrobromides, sulfates, methanesulfonates, nitrates, maleates, acetates, citrates, fumarates, tartrates (e.g., (+)-tartrates, (−)-tartrates, trifluoroacetates, or mixtures thereof including racemic mixtures), succinates, benzoates, and salts with amino acids such as glutamic acid. These salts may be prepared by methods known to those skilled in the art.

The neutral forms of the compounds are preferably regenerated by contacting the salt with a base or acid and isolating the parent compound in the conventional manner. The parent form of the compound differs from the various salt forms in certain physical properties, such as solubility in polar solvents.

In one embodiment the salts are acetate, hydrochloride or trifluoroacetate.

Certain compounds described herein can exist in unsolvated forms as well as solvated forms, including hydrated forms. In general, the solvated forms are equivalent to unsolvated forms and are encompassed within the scope described herein. Certain compounds described herein may exist in multiple crystalline or amorphous forms. In general, all physical forms are equivalent for the uses contemplated herein and are intended to be within the scope described herein.

Certain compounds described herein possess asymmetric carbon atoms (optical centers) or double bonds; and the racemates, diastereomers, tautomers, geometric isomers, and individual isomers are encompassed within the scope described herein. The compounds described herein do not include those compounds known in the art to be too unstable to synthesize and/or isolate.

The compounds described herein may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds described herein, whether radioactive or not, are encompassed within the scope described herein.

The symbol “ [symbol] ” denotes the point of attachment of a chemical moiety to the remainder of a molecule or chemical formula.

As used herein, the singular form “a”, “an”, and “the” includes plural references unless otherwise indicated or clear from context. For example, as will be apparent from context, “a” analog can include one or more analogs. The term “about” in the context of a numeric value refers to +/−10% of the numeric value.

### II. Chemical Synthesis

Reagents. Arachidonic acid was obtained from NU-Check Prep (Elysian, Minn.). All other chemicals and solvents were purchased from Alfa Aesar (Ward Hill, Mass.), Sigma-Aldrich or Fisher Chemicals and were used without further purification. Melting points are reported uncorrected for differences in equipment and conditions. ¹H NMR spectra were recorded on a VMR 500 NMR instrument, and low-resolution mass spectral data were obtained at the Chemistry Department, UCSD. Column chromatography was done on silica gel (Merck grade 230-400 mesh, 60 Å).

Synthetic Chemistry.

The synthetic chemistry followed the general reaction scheme of Nagatsugi et al. (24) modified as described herein. The reactions before the Synthesis of Methyl 20-(tert-butyldimethylsilyloxy)-5,8,11,14-eicosatetraeonate (12) was not reported in their publication.

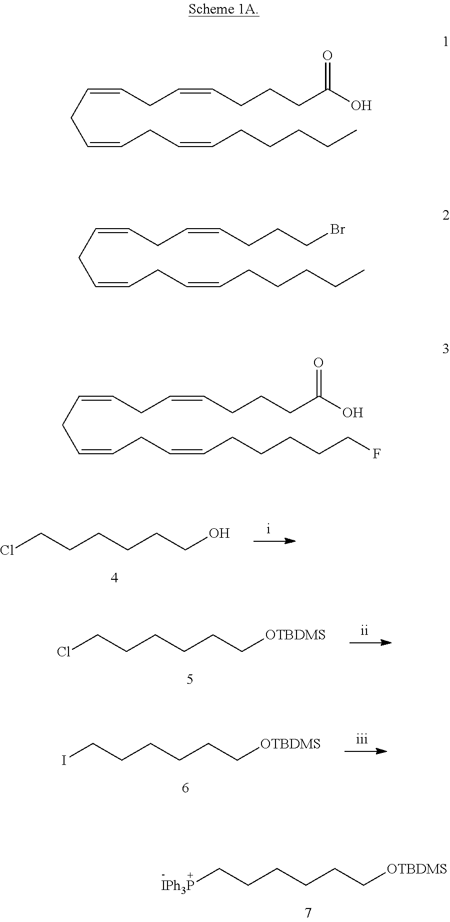

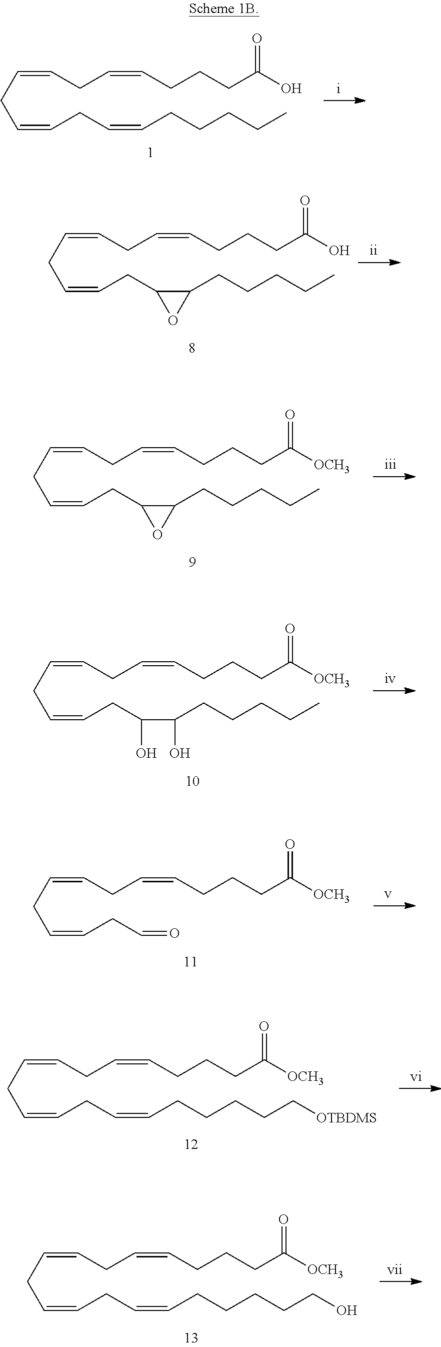

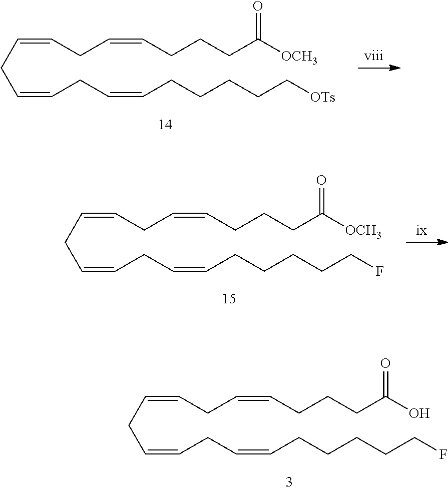 [SMILES: COC(=O)CCC/C=C\C/C=C\C/C=C\C/C=C\CCCCCOS(=O)(=O)c1ccc(C)cc1 ; COC(=O)CCC/C=C\C/C=C\C/C=C\C/C=C\CCCCCF ; O=C(O)CCC/C=C\C/C=C\C/C=C\C/C=C\CCCCCF]

Scheme 1A provides the synthetic route for Wittig salt 7. Conditions: i) TBSCl, imidazole, DMF, DMAP, 0° C.-RT, 15-hr; ii) NaI, acetone, reflux, 10-hr; iii) PPh₃, CH3CN, reflux, 1-hr.

Scheme 1B provides the synthetic routine for 20-fluoroarachidonic acid (20-FAA). Conditions: i) 1,1′ carbonyl diimidazole, H₂O₂, imidazole, KHSO₄, DCM, 0° C.-RT, 40-min; ii) 1,1′ carbonyl diimidazole, MeOH, DCM, 2-hr, 0° C.-RT; iii) 10% HClO₄, THF, 0° C.-RT, 4-hr; iv) Pb(OAc)₄, DCM, −20° C., 30-min; v) n-BuLi, Cmpd 7 Wittig salt, THF, −78° C., 3-hr; iv) TBAF, THF, 0° C.-RT, 3-hr; vii) TsCl, Et₃N, DCM, 0° C.-RT, 6-hr; viii) TBAF, THF, 0° C.-RT; ix) LiOH, THF:H₂O, RT, 24-hr.

### Synthesis of tert-butyl(6-chlorohexyloxy)dimethylsilane (5)

To a stirred solution of alcohol 4 (10 g, 73 mmol) in dimethylformamide (DMF 40 mL), imidazole (5.48 g, 80 mmol) and tert-butyldimethylchlorosilane (TBDMSCl 12.13 g, 80 mmol) were added sequentially at 0° C. under N₂, before addition of a catalytic amount of DMAP (0.09 g, 7.3 mmol) to the reaction mixture. After stirring for 15 h at room temperature, the reaction mixture was quenched with saturated NH₄Cl (20 mL) solution and extracted with CH₂Cl₂ (100 mL). The organic extract was dehydrated with Na₂SO₄ and concentrated in vacuo. Purification by column chromatography (SiO₂, 1% EtOAc in hexane eluant) gave pure compound (16.3 g, 89%) as clear oil 5.

### Synthesis of tert-butyl(6-iodohexyloxy)dimethylsilane (6)

To a stirred solution of tert-butyl(6-chlorohexyloxy)dimethylsilane 5 (5 g, 19.9 mmol) in acetone (50 mL), NaI (14.9 g, 99.6 mmol) was added at room temperature and the reaction mixture was refluxed for 10 h. The acetone solvent was evaporated in vacuo to obtain a residue, which was redissolved in CH₂Cl₂ and washed sequentially with Na₂S₂O₃, water and finally with saturated brine solution. The organic extract was dried with Na₂SO₄, concentrated in vacuo and purified by column chromatography (SiO₂, 1% EtOAc in hexane eluant) to give a pure compound (6.5 g, 95%) as a light yellow oil 6.

### Synthesis of (6-(tert-butyldimethylsilyloxy)hexyl)triphenylphosphonium iodide (7)

A mixture of triphenylphosphine (3.8 g, 14.6 mmol) and iodo compound 6 (5 g, 14.6 mmol) in acetonitrile (50 mL) was refluxed for 10 h under N₂ atmosphere. The mixture was cooled to room temperature and concentrated in vacuo. The pure Wittig salt was obtained by the purification of the residue using column chromatography (SiO₂, 3% MeOH in dichloromethane as eluant) as a gummy substance 7 (8.2 g, 93%).

### Synthesis of 14,15-Epoxyeicosa-cis-5,8,11-trienoic acid (8)

To a stirred solution of arachidonic acid (4.4 mL, 13.3 mmol) dissolved in anhydrous CH₂Cl₂ (60 mL), 1,1′-carbonyldiimidazole (2.26 g, 14.0 mmol) was added under N₂ at 0° C. After stirring for 40 min, the reaction mixture was added to ethereal H₂O₂ solution (135 mL, 560 mmol) containing catalytic amount of lithiumimidazole (16 mg) at 0° C. KHSO₄ (26 g, 190 mmol) was added after the addition of half of the arachidonyl imidazolide solution and the resulting mixture was stirred for an additional 5 min. The reaction mixture was filtered, washed with water, and brine, and dried over Na₂SO₄ overnight. Solvent was evaporated under reduced pressure to obtain the title compound as viscous oil 8 (3.8 g), which was used for next reaction without any further purification.

### Synthesis of Methyl 14,15-Epoxyeicosa-cis-5,8,11-trienoate (9)

To a solution of 1,1′-carbonyldiimidazole (2.5 g, 15.9 mmol) in anhydrous CH₂Cl₂ (50 mL), epoxy arachidonic acid (3.8 g) 8 was added under N₂ at 0° C. MeOH (5 mL) was added after stirring for 30 min, and the reaction was stirred for another 4 h at room temperature. The solvent was concentrated in vacuo and the residue was redissolved in ether and washed with water, brine and dried over Na₂SO₄. After evaporation of the solvent, purification of the residue by column chromatography (SiO₂, 10% EtOAc in hexane as eluant) gave pure title compound as a viscous oil 9 (1.4 g, 55%).

### Synthesis of Methyl 14,15-Dihydroxyeicosa-cis-5,8,11-trienoate (10)

To a stirred solution of epoxy ester (1.1 g, 3.2 mmol) in THF (20 mL), was added cold 10% HClO₄ (9.1 mL) at 0° C. The reaction mixture was stirred for another 4 hours followed by quenching with aqueous NaHCO₃ and extracted with ethyl acetate, washed with water, brine and dried over Na₂SO₄, filtered and concentrated in vacuo to obtain crude compound. Purification by column chromatography (SiO₂, hexane—20% EtOAc in hexane as eluant) afforded the title compound as a viscous oil 10 (0.78 g, 68%).

### Synthesis of Methyl 20-(tert-butyldimethylsilyloxy)-5,8,11,14-eicosatetraeonate (12)

Compound 12 was prepared by Wittig reaction according to the modified procedure of Nagatsugi et al. (24). The crude compound was purified by column chromatography (SiO₂, Hexane—2% EtOAc in hexane as eluant) to afford pure title compound as colorless oil 12. ¹H NMR (400 MHz, CDCl₃) analysis provided the following results: δ 0.05 (s, 6H), 0.89 (s, 9H), 1.50-1.21 (m, 6H), 1.76-1.66 (m, 2H), 2.27-2.03 (m, 4H), 2.32 (t, J=7.6 Hz, 2H), 2.86-2.78 (m, 6H), 3.60 (t, J=6.6 Hz, 2H), 3.67 (s, 3H), 5.45-5.30 (m, 8H).

### Synthesis of Methyl 20-hydroxy-5,8,11,14-eicosatetraenoate (13)

To a stirred solution of tert-butyldimethylsilyl (TBS) protected alcohol 12 (0.051 g, 0.11 mmol) in anhydrous THF (2 mL), tetrabutylammonium fluoride (0.17 mL, 0.17 mmol, 1M THF solution) was added at 0° C. under N₂ atmosphere. The reaction mixture was warmed to room temperature and stirred for 5 h. It was quenched with saturated aqueous NH₄Cl solution, extracted with EtOAc, washed with brine, dried (Na₂SO₄) and concentrated in vacuo. Purification by column chromatography (SiO₂, hexane—10% EtOAc in hexane as eluant) afforded the title compound as a clear oil 13 (0.036 g, 96%). ¹H NMR (400 MHz, CDCl₃) analysis revealed the following information: δ 1.27 (br, 1H), 1.60-1.36 (m, 6H), 1. 1.68-2.15 (m, 2H), 2.33 (t, J=7.6 Hz, 2H), 2.86-2.78 (m, 6H), 3.60 (t, J=6.6 Hz, 2H), 3.67 (s, 3H), 5.45-5.30 (m, 8H). ¹³C NMR (100 MHz, CDCl₃) δ 24.6, 25.3, 25.4, 26.4, 27.0, 29.1, 29.3, 30.2, 32.5, 33.3, 51.3, 62.6, 127.6, 127.8, 128.3, 128.5, 128.7, 128.8, 130.0, 130.8.

### Synthesis of Methyl 20-(p-toluenesulfonyloxy)-5,8,11,14-eicosatetraenoate (14)

To a stirred solution of alcohol 13 (0.04 g, 0.11 mmol) in anhydrous CH₂Cl₂ (2 mL), triethylamine (0.33 mL, 0.22 mmol) and p-toluenesulfonyl chloride (0.041 g, 0.22 mmol) were added sequentially at 0° C. under N₂ atmosphere. The reaction mixture was warmed to room temperature and stirred for another 6 h. Ice cold water was added, extracted with water, washed with water and saturated brine, dried (with Na₂SO₄), filtered and concentrated under the reduced pressure. Purification by column chromatography (SiO₂, hexane—2% EtOAc in hexane as eluant) gave the compound (49.3 mg, 93%) as colorless oil 14. ¹HNMR (400 MHz, CDCl₃) δ 7.79 (d, J=8.4 Hz, 2H), 7.35 (d, J=8.4 Hz, 2H), 5.35 (m, 8H), 4.01 (t, J=6.0 Hz, 2H), 3.65 (s, 3H), 2.81 (m, 6H), 2.31 (t, J=7.5 Hz, 2H), 2.11 (m, 4H), 1.71 (p, J=7.2 Hz, 2H), 1.62 (m, 2H), 1.39 (m, 4H); ¹³C NMR (100 MHz, CDCl₃) δ 21.5, 24.6, 25.2, 25.5, 26.4, 26.8, 27.0, 28.6, 30.2, 33.3, 51.4, 70.4, 127.7, 127.9, 127.9, 128.0, 128.1, 128.7, 128.8, 129.6, 129.7, 133.0, 144.5, 173.9.

### Synthesis of Methyl 20-fluoroarachidonate (15)

To a stirred solution of tosylate 14 (0.04 g, 0.089 mmol) in THF (1 mL), tetrabutylammonium fluoride (0.35 mL, 0.35 mmol, 1M THF solution) was added at 0° C. under N₂ atmosphere. The reaction mixture was stirred for 2 h followed by the evaporation of solvent under reduced pressure. This gave a crude compound, which was redissolved in CH₂Cl₂, washed with water, brine, dried with Na₂SO₄, filtered and concentrated in vacuo. Purification of column chromatography (SiO₂, hexane—2% EtOAc in hexane as eluant) afforded title compound as a pale yellow oil 15 (0.028 g, 95%). ¹H NMR (400 MHz, CDCl₃) indicated the following results: δ 5.38 (m, ═CH), 4.5 (dt, J=47.0, 6 Hz, CH2F), 4.38 (dt, J=47.0, 6 Hz, CH2F), 3.67 (s, OCH3), 2.82 (d, J=2.8 Hz, CH2), 2.33 (t, J=7.6 Hz, CH2), 2.12 (m, CH2), 1.73 (m, CH2), 1.42; 13C NMR δ 24.75, 24.83, 25.60, 26.53, 27.06, 29.20, 29.67, 30.20, 30.39, 30.2, 30.4, 33.4, 51.44, 83.25, 84.88, 127.97, 128.15, 128.19, 128.44, 128.76, 129.0, 129.95, 174.02.

### Synthesis of 20-Fluoroarachidonic acid (3)

To a stirred solution of ester 15 (0.025 g, 0.074 mmol) in THF:water (5:1), LiOH was added at 0° C. under N₂ atmosphere. The resulting mixture was stirred for 24 h at room temperature. The mixture was quenched with 1M aqueous oxalic acid and the solvent was removed under reduced pressure, extracted with CH₂Cl₂, washed with water, brine, dried over Na₂SO₄, filtered and evaporated under reduced pressure to obtain a crude acid. Purification by column chromatography (SiO₂, hexane—20% EtOAc in hexane as eluant) gave pure acid as colorless oil 3 (0.22 g, 95%). ¹H NMR (400 MHz, CDCl₃) analysis indicated the following: δ 5.38 (m, ═CH), 4.5 (dt, J=47.2, 6 Hz, CH2F), 4.38 (dt, J=47.2, 6 Hz, CH2F), 2.82 (m, CH2), 2.38 (t, J=7.6 Hz, CH2), 2.12 (p, J=7.6 Hz, CH2), 1.73 (m CH2), 1.42; 13C NMR (100 MHz, CDCl3) δ 24.9, 24.8, 24.8, 25.6, 26.4, 27.0, 29.2, 29.7, 30.2, 30.2, 30.2, 30.4, 33.3, 83.2, 84.9, 127.97, 128.15, 128.19, 128.44, 128.76, 129.0, 129.95, 179.52; ESI MS: m/z 321.46(M-H+).

### III. Pharmacokinetics

Animals.

The plasma and brain kinetics of the purified [¹⁹F]FAA were tested in unanesthetized adult C57BL/6J male mice. The study was conducted following the National Institutes of Health Guidelines for the Care and Use of Laboratory Animals (Publication no. 80-23) and was approved by the Animal Care and Use Committee of the Eunice Kennedy Shriver National Institute of Child Health and Human Development. Male C57BL/6J mice were obtained at the age of 2-3 months. The mice were maintained in an animal facility in which temperature, humidity, and light cycle were regulated with free access to water and a fixed diet (Rodent NIH-07) that contained (as fractional percent of total fatty acids), 30.6% saturated, 22.5% monounsaturated, 47.1% linoleic, 4.9% α-linolenic, 0.2% AA, 1.6% eicosapentaenoic, and 2.2% docosahexaenoic acid.

Surgical Procedures and Tracer Infusion.

At 4-5 months of age, a mouse was anesthetized with 1-3 isofluorane, and polyethylene catheters were inserted into the femoral artery and vein. The mouse was allowed to recover from surgery in an environment maintained at 25° C., with its hindquarters loosely wrapped and taped to a wooden block. During recovery, body temperature was maintained at 37° C. by means of a rectal probe and a heating element (Indicating Temperature Controller; Yellow Springs Instrument). After 3 h of recovery, the unanesthetized mouse was infused intravenously for 5 min with 130 μl HEPES buffer (pH 7.4) containing 50 mg/ml fatty acid-free bovine serum albumin (BSA, Sigma), or 0.1 mg or 0.5 mg 20-fluoroarachidonic acid (20-FAA) dissolved in HEPES-BSA at a rate of 0.0223 (1+e^{-0.32t}) ml/min (t in seconds), using a computer-controlled variable rate infusion pump (No. 22; Harvard Apparatus). During infusion, timed arterial blood samples (˜20 μl) were collected in polyethylene-heparin lithium fluoride-coated Beckman centrifuge tubes at 0, 0.25, 0.5, 1.0, 1.5, 3.0, and 4.0 min, and 150 μl was collected at 4.9 min. Plasma samples were separated by centrifuging at 13,000 rpm for 1 min, and stored at −80° C. until they were analyzed. At 5 min, the mouse was anesthetized with sodium pentobarbital (50 mg/kg, i.v.) and subjected to head-focused microwave irradiation (5.5 kW, 0.9 s, 75% power output; Cober Electronics) to stop brain fatty acid metabolism (27). The brain was excised, dissected sagittally and stored at −80° C. until analysis.

Plasma and Brain Lipid Extraction and Separation.

Total lipids were extracted from plasma (7-10 μl) and from half brain (˜0.2 g) by the method of Folch et al (28). Heptadecanoic acid (17:0) was added as an internal standard to plasma prior to extraction. Total lipid extracts were separated by thin layer chromatography (TLC) on silica gel plates (Silica Gel 60A TLC plates; Whatman) that were pre-washed with chloroform/methanol (2:1 v/v) and activated by heating at 100° C. Neutral lipid subclasses including phospholipids (PLs), unesterified fatty acids (UFAs), triglycerides (TGs) and cholesteryl esters (CEs) were separated using a mixture of heptane:diethyl ether:glacial acetic acid (60:40:3 by volume) (29), and authentic PL, cholesterol, UFA, TG and CE standards were run in separate lanes to identify the bands. Phospholipid classes (EtnGpl, ethanolamine glycerophospholipid; ChoGpl, choline glycerophospholipid; PtdIns, phosphatidylinositol; PtdSer, phosphatidylserine) were separated in chloroform:methanol:H₂O:glacial acetic acid (60:50:4:1 by volume) (30) and identified with authentic standards in separate lanes. The plates were sprayed with 0.03% (w/v) 6-p-toluidine-2-naphthalene sulfonic acid (Acros) in 50 mM Tris-HCl buffer (pH 7.4), and the lipid bands were visualized under UV light. Each band was scraped into tubes containing di-17:0 PC for quantifying PL, CE, TG, EtnGpl, ChoGpl, PtdIns and PtdSer lipid fractions, or 17:0 heptadecaenoic acid to quantify brain UFAs. Toluene (0.2 ml) was added to each tube. The UFAs were extracted from the scraped silica using 2:1 chloroform/methanol and 1.5 ml of 0.1 M KCl, dried and derivitized as described below. The remaining silica extracts containing esterified fatty acids (PL, CE, TG, EtnGpl, ChoGpl, PtdIns and PtdSer) were hydrolyzed with 1 ml of 10% methanolic KOH at 70° C. for one hour and the resulting free fatty acids were displaced from with 1 ml HCl (37%) and 1 ml H₂O, and extracted with 3 ml hexane. The top hexane layer was transferred to new tubes following vortexing and centrifugation at 3000 rpm (5 min), and subjected to derivatization as described below.

Derivatization.

The hydrolyzed fatty acids (in hexane) were dried and derivatized in 100 μl pentafluorobenzyl (PFB) bromide (Sigma-Aldrich) reagent containing acetonitrile, diisopropylamine and PFB (1:100:10 v/v/v), by shaking for 15 minutes. The PFB reagent was dried and the derivatized fatty acids were dissolved in 50-100 μl hexane and transferred to vials for gas-chromatography-mass spectrometry (GC/MS) analysis.

GC/MS Analysis.

GC/MS analysis was performed using a Finnigan TRACE DSQ mass spectrometer (Thermo Electron) coupled with TRACE GC. The fatty acyl PFB esters in hexane were injected onto a DB-FFAP capillary column (30 m×0.25 mm i.d., 0.25 μm film thickness, J&W Scientific, Folsom, Calif.) interfaced directly into the negative chemical ionization source using methane as reagent gas and helium as carrier gas. The GC column oven temperature was programmed from 80° C. to 185° C. at 20° C. per min, then to 240° C. at 10° C. per min and held for 30 min. The injector and transfer lines were maintained at 240° C. and 220° C., respectively. The 17:0 internal standard and FAA were monitored by selected ion mode (SIM) of the base peak (M-PFB). The concentration of FAA was quantified by relating its peak area to the area of the internal standard and correcting for the response factor, which was determined from a calibration curve of the FAA standard. The response factor for FAA was 0.2.

Calculations.

Based on the intravenous infusion of [¹⁹F]FAA, incorporation coefficients into individual brain phospholipids, k*_{I}(ml/s/g×10⁻⁴), were calculated as follows (26):

$${k}_{i}^{*} = \frac{{c}_{brain , i}^{*} ( T )}{{\int }_{0}^{T} {c}_{plasma}^{*} d t}$$

wherein, C*_{brain} (nmol·g⁻¹) is the FAA concentration in brain lipid i at time T=5 min (time of termination of experiment), t is time after starting infusion, and c*ₚₗₐₛₘₐ nmol·ml⁻¹ is plasma concentration of unesterified FAA during infusion. Integrals of plasma radioactivity were determined by trapezoidal integration.

### IV. Results and Discussion

Chemical Synthesis.

20-[¹⁹F]FAA 3 was prepared from readily available arachidonic acid by an improvement of the reported procedure (24). Arachidonic acid 1 has four (Z,Z) double bonds at Δ5,6-, Δ8,9-, Δ11,12- and the Δ14,15-positions. Construction of the Z-stereochemistry in Δ14,15 olefin of arachidonic acid is essential for the preparation of 20-FAA. This investigation suggested that it may be possible to prepare fluoroarachidonic acid 3 of established geometry by cleaving Δ14,15-olefin employing reagents which do not react at the Δ5,6-, Δ8,9-, Δ11,12-olefin site of the arachidonic acid.

The m-chloroperbenzoic acid (mCPBA) used previously is unsuitable for selective epoxidation of arachidonic acid. The selective epoxidation of arachidonic acid was carried out by two different methods. First, the reaction of arachidonic acid with oxalyl chloride in benzene afforded acid chloride. Oxidation of acid chloride with 50% hydrogen peroxide in the presence of pyridine and lithium hydroxide gave peroxy arachidonic acid. This acid was rearranged to the epoxy acid 8. The epoxy acid was obtained in 25% yield. This method was not satisfactory for the preparation of epoxy acid due to the low yield, poor scale up and difficulty in handing the acid chloride.

We then investigated another method in which the arachidonic acid was oxidized with a dry solution of hydrogen peroxide in diethyl ether-dichloromethane and carbonyldiimidazole in the presence of a basic catalyst lithiumimidazole to form peroxy arachidonic acid, which is selectively converted to 14,15-epoxyarachidonic acid 8 (31). This intramolecular epoxidation of peroxy arachidonic acid generated the 14,15-epoxide with high efficiency. The selective formation of epoxy acid 8 from arachidonic acid 1 can be ascribed to the stereoelectronic constraints for intramolecular oxygen transfer arising from attack by the C═C pi orbital electrons backside to the O—O bond (sigma orbital) of the internally hydrogen bonded peroxycarbonyl group with the C═C sigma plane approximately perpendicular to the peroxycarbonyl ring. The selectivity is arguably controlled by the ring strain because epoxidation requires a perpendicular orientation between C═C sigma plane and the internally hydrogen-bonded peroxy carbonyl ring. Epoxidation of the other three C═C double bonds Δ5,6 olefin, Δ8,9 olefin and Δ11,12 olefin requires more ring strain, thus is less favored.

Of the two methods described above which led to the epoxy acid, the latter approach described in scheme 1 is more versatile than the former one based on the acid chloride reaction. Preparation of the epoxy ester via acid chloride and anhydride is ruled out because of the labile functional moiety, the epoxy group. There are two common methods to prepare the epoxy methyl ester 9. First, the epoxy acid 8 was converted to the methyl ester by diazomethane treatment. This reagent was prepared in ethereal solution by the action of sodium hydroxide with N-methyl-N-nitroso-p-toluene-sulfonamide (Diazald™, Aldrich Chemical Co.) in the presence of an alcohol. This procedure is very difficult to scale up to gram quantities. Diazomethane is very explosive and toxic. As a result, we did not continue to make epoxy methyl ester with diazomethane in large scale. A useful alternative, which gives high yields for fatty acid components (32), is to activate the latter by reacting with 1,1′-carbonyldiimidazole to form an imidazolide, which is reacted immediately (without isolation) with methanol in the presence of a base to give the required ester 9. This procedure has been used in the preparation of epoxy methyl ester. Observation of the thin layer chromatography of methyl ester indicated the absence of the epoxy acid 8.

The epoxide group of 9 was cleaved to a diol 10 using perchloric acid in a mixture of THF and water in 55% yield. The diol 10 that was cleaved with lead tetraacetate to furnish the aldehyde 11 in low yield.

A literature bromination or iodination reaction for cross coupling with this aldehyde 11 in order to construct the C₁₆-C₂₀ carbon side chain in 20-FAA was carried out without success. (33) (34).

However another approach was successful. The commercially available 6-chlorohexanol 4 was protected as its tert-butyldimethylsilyl ether. The compound 5 was obtained in 89% yield. After the protection of the hydroxyl group with tert-butyldimethylsilyl group, the chloro compound 5 was heated to react with sodium iodide in anhydrous acetone to give an iodo compound 6 in 95% yield. The ylides themselves are generated in a two-step sequence which involves nucleophilic attack of triphenylphosphine on iodo compound 6 to form a phosphonium salt 7. A five-carbon extension of the labile aldehyde intermediate was carried out by the Wittig reaction. This Wittig salt 7 was treated with a base n-butyllithium to generate the ylide. The Wittig salt was thus allowed to react with 2 equiv of n-butyllithium at −78° C. to ensure the formation of the ylide. The resultant ylide solution was treated with labile aldehyde 11 as shown in scheme 1. The Wittig product was obtained as a mixture of major Z-stereochemistry in Δ14,15 olefin and the minor E-stereochemistry Δ14,15 olefin as very low yield. The attempts to separate the two Δ14,15 olefin cis and trans isomers by routine column chromatography were unsuccessful.

The TBDMS group of the 12 was then desilylated using tetrabutylammonium fluoride to provide an alcohol 13 in 96% yield. As a result, the two isomers the major Z-stereochemistry in Δ14,15 olefin and the minor E-stereochemistry in Δ14,15 olefin were easily separated by flash chromatography. The stereochemistry of the isolated 13 was determined by NMR spectroscopy on the 14-15 double bonds. Only the Z isomer was detected by proton NMR spectroscopy. The alcohol was not contaminated with the trans isomer. As expected, the Δ14,15 E-alkene isomer was not detected by proton NMR spectroscopy and carbon NMR spectroscopy.

Generally, triflate precursors are unstable and thus difficult to isolate and purify. The mesylate precursor is considered to be a more stable precursor than the tosylate. But tosylation of 13 with TsCl in pyridine gave no better results. On the other hand, tosylation of 13 with TsCl in triethylamine gave the tosylate 14 in 93% yield. Nucleophilic displacement of the tosylate 14 with tetrabutylammonium fluoride (TBAF) in THF furnished the desired fluoro compound 15 in 95% yield. Careful treatment of 15 with 3 equiv of lithium hydroxide at 0° C. to ambient temperature selectively hydrolyzed the methyl ester, which was purified by column chromatography to furnish 20-FAA al in 95% yield (Scheme 1). Based on literature precedent, the tosylate or mesylate precursor can be used to produce 20-[¹⁸F]FAA in sufficient yields in reaction times and temperature commensurate with the half life and stability of the precursor (35).

Pharmacokinetic Studies in Mice.

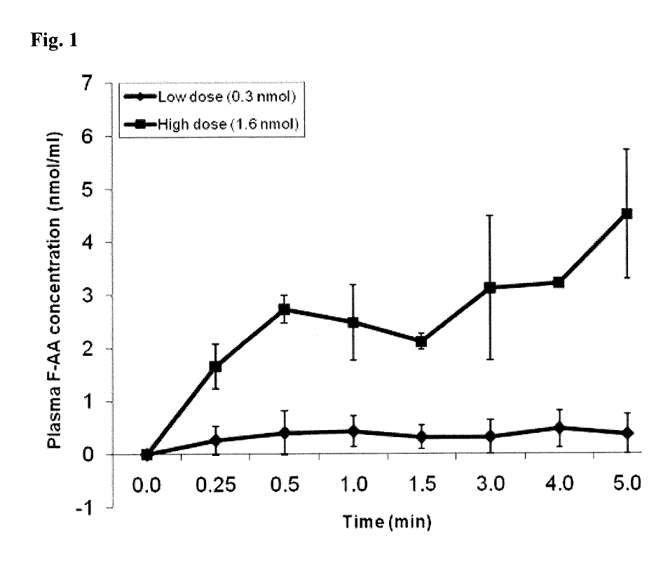

The in vivo pharmacokinetics of FAA were determined in mice during a 5-minute intravenous infusion of a low FAA dose (0.3 nmol) equivalent to the [¹⁴C]AA dose used in a previous study (2), or a high dose (1.6 nmol). The FAA concentration in plasma (nmol/ml) increased over time in a dose-dependent manner (FIG. 1). The plasma area under the curve for the low and high dose was 107±78 nmol s ml⁻¹ and 849±68 nmol s ml⁻¹, respectively. Statistical analysis was not performed due to the small sample sizes (n=2 to 3).

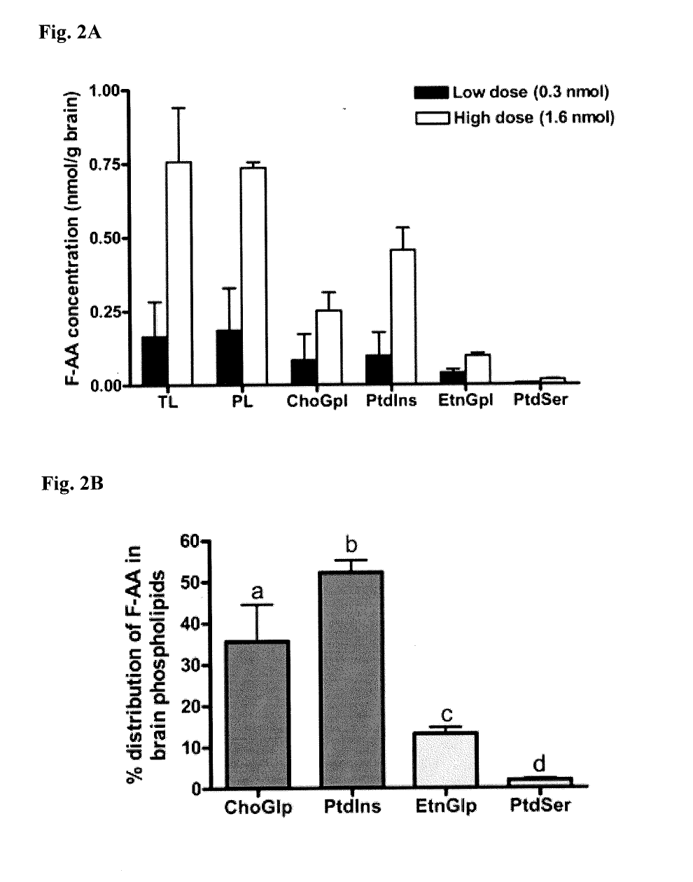

Nagatsugi et al. reported the incorporation of a 20-[¹⁸F]FAA injected brain total lipid extracts, but did not confirm its presence in brain phospholipids (25). In our study, GC/MS analysis revealed the presence at the end of the 5-min infusion of [¹⁹F]FAA in brain total lipids, total phospholipid and individual phospholipid fractions, in a dose-dependent manner (FIG. 2-A). Most FAA was detected in phospholipid and none or a negligible amount (<0.01 nmol/g) was detected in the brain unesterified fatty acid, triglyceride and cholesteryl ester fractions. Within phospholipids, FAA concentration was highest in PtdIns and ChoGpl, followed by EtnGpl and PtdSer, for both doses (FIG. 2-A). FAA was not detected in brain lipids of the saline-albumin infused control mouse.

FIG. 2-B shows the distribution of FAA in each phospholipid fraction, expressed as a percentage of the FAA concentration in total phospholipid (i.e., % nmol FAA in each PL fraction/nmol FAA in total PL). The mean percentages differed significantly from each other, being the highest in ChoGlp, followed by PtdIns, EtnGlp and PtdSer (p<0.01). This suggested preferential accumulation of FAA in PtdIns and ChoGpl.

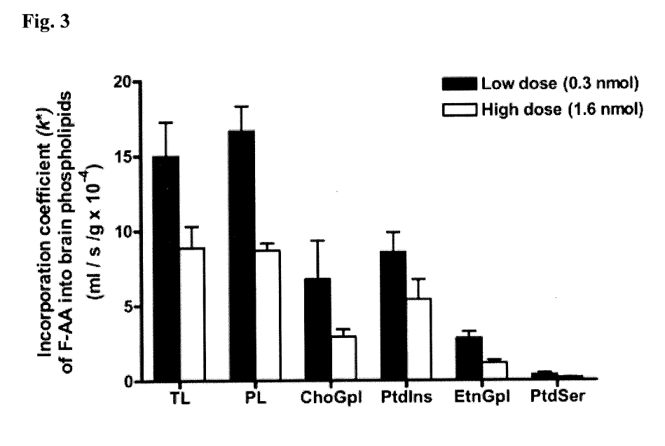

The incorporation coefficients, k* (Eq. 1), for both low and high doses are shown in FIG. 3. As indicated, FAA was incorporated into brain total lipids and phospholipids, and in individual phospholipids. k* was lower for the high infusion dose than the low dose, suggesting substrate inhibition at the higher dose. Within PLs, FAA was incorporated preferentially into PtdIns and ChoGpl at both doses (FIG. 3). This preferential incorporation is similar to the preferential incorporation of [¹⁴C]AA (1).

Table 1 following compares mean values of k* for FAA in our study to published values for intravenously injected [¹⁴C]AA in mice (2). For Table 1, values are mean±SD (n=3 mice for [¹⁹F-FAA]. For both tracers, values of k* into individual phospholipids are comparable, and higher in PtdIns and ChoGpl than EtnGpl and PtdSer (1, 2). These results indicate that adding ¹⁹F to AA at the 20 position did not markedly change its incorporation kinetics or enzyme or phospholipid selectivities compared with the natural substance. Enzymes involved incorporation are acyl-CoA synthetases, which activate AA to AA-CoA, and acyl CoA: lysophospholipid acyltransferase, which transfer AA from AA-CoA into an open sn-2 position site of a lysophospholipid (26, 36, 37). These enzymes show selectivities for AA compared with other fatty acids (38, 39).

TABLE 1

| | |
|---|---|
| k* values for | Published k* values for [1- |
| [¹⁹F]FAA | ¹⁴C]AA in mouse¹ |

| | |
|---|---|
| Brain lipid | ml/s/g × 10⁴ |

| | | |
|---|---|---|
| Total lipid | 15.0 ± 2.3 | 22.6 ± 1.3 |
| ChoGpl | 6.8 ± 2.6 | 5.9 ± 1.4 |
| PtdIns | 8.6 ± 1.3 | 6.8 ± 0.8 |
| PtdSer | 0.4 ± 0.1 | 1.8 ± 0.5 |
| EtnGpl | 2.8 ± 0.4 | 5.7 ± 1.0 |

¹From (2).

### V. Summary

In summary, we developed a stereoselective synthesis of non-radioactive [¹⁹F]FAA yield, which was incorporated preferentially into mouse brain PtdIns and ChoGpls, as has been reported for [¹⁴C]AA, and with similar coefficients. This development can expand on present PET imaging capabilities, allowing centers to synthesize using the disclosed method positron-emitting [¹⁸F]FAA for PET imaging of upregulated brain AA metabolism in clinical inflammation, which likely occurs in Alzheimer's disease, bipolar disorder, schizophrenia and Parkinson's disease and also in HIV-1 associated dementia, brain trauma, epilepsy, cancer and bacterial infections (40, 41) and rheumatoid arthritis, inflammatory bowel disease, and metabolic syndrome. A longer radioactive half-life than that of [¹¹C]AA would also enhance current resolution limitations of PET scans, since more radioactivity can be captured and integrated over time to obtain a higher resolution image. 20-[¹⁸F]FAA has the potential to be an important radio-biomarker in patient management and assessment in neurology, psychiatry, oncology, and cardiology.

### VI. References

1. DeGeorge J J, Noronha J G, Bell J, Robinson P, Rapoport S I. Intravenous injection of [1-14C]arachidonate to examine regional brain lipid metabolism in unanesthetized rats. J Neurosci Res. November 1989; 24(3):413-423, incorporated by reference herein in its entirety.
2. Rosenberger T A, Villacreses N E, Contreras M A, Bonventre J V, Rapoport S I. Brain lipid metabolism in the cPLA2 knockout mouse. J Lipid Res. January 2003; 44(1):109-117, incorporated by reference herein in its entirety.
3. Basselin M, Fox M A, Chang L, et al. Imaging elevated brain arachidonic acid signaling in unanesthetized serotonin transporter (5-HTT)-deficient mice. Neuropsychopharmacology. June 2009; 34(7):1695-1709, incorporated by reference herein in its entirety.
4. Basselin M, Ramadan E, Igarashi M, et al. Imaging upregulated brain arachidonic acid metabolism in HIV-1 transgenic rats. J Cereb Blood Flow Metab. February 2011; 31(2):486-493, incorporated by reference herein in its entirety.
5. Dirnagl U, Iadecola C, Moskowitz M A. Pathobiology of ischaemic stroke: an integrated view. Trends Neurosci. September 1999; 22(9):391-397, incorporated by reference herein in its entirety.
6. Esposito G, Giovacchini G, Liow J S, et al. Imaging neuroinflammation in Alzheimer's disease with radiolabeled arachidonic acid and PET. J Nucl Med. September 2008; 49(9):1414-1421, incorporated by reference herein in its entirety.
7. Minghetti L. Role of inflammation in neurodegenerative diseases. Curr Opin Neurol. June 2005; 18(3):315-321, incorporated by reference herein in its entirety.
8. Rao J S, Harry G J, Rapoport S I, Kim H W. Increased excitotoxicity and neuroinflammatory markers in postmortem frontal cortex from bipolar disorder patients. Mol Psychiatry. April 2010; 15(4):384-392, incorporated by reference herein in its entirety.
9. Basselin M, Chang L, Bell J M, Rapoport S I. Chronic lithium chloride administration attenuates brain NMDA receptor-initiated signaling via arachidonic acid in unanesthetized rats. Neuropsychopharmacology. August 2006; 31(8):1659-1674, incorporated by reference herein in its entirety.
10. Basselin M, Villacreses N E, Lee H J, Bell J M, Rapoport S I. Chronic lithium administration attenuates up-regulated brain arachidonic acid metabolism in a rat model of neuroinflammation. J. Neurochem. August 2007; 102(3):761-772, incorporated by reference herein in its entirety.
11. Ramadan E, Basselin M, Taha A Y, et al. Chronic valproate treatment blocks D(2)-like receptor-mediated brain signaling via arachidonic acid in rats. Neuropharmacology. December 2011; 61(8):1256-1264, incorporated by reference herein in its entirety.
12. Rao J S, Kim H W, Kellom M, et al. Increased neuroinflammatory and arachidonic acid cascade markers, and reduced synaptic proteins, in brain of HIV-1 transgenic rats. J. Neuroinflammation. 2011; 8:101, incorporated by reference herein in its entirety.
13. Esposito G, Giovacchini G, Der M, et al. Imaging signal transduction via arachidonic acid in the human brain during visual stimulation, by means of positron emission tomography. Neuroimage. Feb. 15 2007; 34(4):1342-1351, incorporated by reference herein in its entirety.
14. Giovacchini G, Lerner A, Toczek M T, et al. Brain incorporation of 11C-arachidonic acid, blood volume, and blood flow in healthy aging: a study with partial-volume correction. J Nucl Med. September 2004; 45(9):1471-1479, incorporated by reference herein in its entirety.
15. Chang M C, Arai T, Freed L M, et al. Brain incorporation of [1-11C]arachidonate in normocapnic and hypercapnic monkeys, measured with positron emission tomography. Brain Res. Apr. 25 1997; 755(1):74-83, incorporated by reference herein in its entirety.
16. Channing M A, Simpson N. Radiosynthesis of 1-[11C]polyhomoallylic fatty acids. Labelled Compds Radiopharm 1993; 33:541-546, incorporated by reference herein in its entirety.
17. Lajis N H, Khan M N, Hassan H. Evidence for the Occurrence of Substitution Side Products in Grignard Reactions. Tetrahedron 1993; 49:3405-3410, incorporated by reference herein in its entirety.
18. Alavi A, Dann R, Chawluk J, Alavi J, Kushner M, Reivich M. Positron emission tomography imaging of regional cerebral glucose metabolism. Semin Nucl Med. January 1986; 16(1):2-34, incorporated by reference herein in its entirety.
19. Chawluk J B, Alavi A, Dann R, et al. Positron emission tomography in aging and dementia: effect of cerebral atrophy. J Nucl Med. April 1987; 28(4):431-437, incorporated by reference herein in its entirety.
20. Gur R E, Resnick S M, Alavi A, et al. Regional brain function in schizophrenia. I. A positron emission tomography study. Arch Gen Psychiatry. February 1987; 44(2):119-125, incorporated by reference herein in its entirety.
21. Kushner M, Tobin M, Alavi A, et al. Cerebellar glucose consumption in normal and pathologic states using fluorine-FDG and PET. J Nucl Med. November 1987; 28(11):1667-1670, incorporated by reference herein in its entirety.
22. Silverman D H, Small G W, Chang C Y, et al. Positron emission tomography in evaluation of dementia: Regional brain metabolism and long-term outcome. JAMA. Nov. 7 2001; 286(17):2120-2127, incorporated by reference herein in its entirety.
23. Krohn K A, Mankoff D A, Muzi M, Link J M, Spence A M. True tracers: comparing FDG with glucose and FLT with thymidine. Nucl Med Biol 2005; 32:663-671, incorporated by reference herein in its entirety.
24. Nagatsugi F, Hokazono J, Sasaki S, Maeda M. Synthesis of 20-[18F]fluoroarachidonic Acid: A potential phospholipid metabolic agent. J Labelled Compds Radiopharm. 1994; 34:1121-1127, incorporated by reference herein in its entirety.
25. Nagatsugi F, Hokazono J, Sasaki S, Maeda M. 20-[¹⁸F]fluoroarachidonic acid: tissue biodistribution and incorporation into phospholipids. Biol Pharm Bull. October 1996; 19(10):1316-1321, incorporated by reference herein in its entirety.
26. Robinson P J, Noronha J, DeGeorge J J, Freed L M, Nariai T, Rapoport S I. A quantitative method for measuring regional in vivo fatty-acid incorporation into and turnover within brain phospholipids: review and critical analysis. Brain Res Brain Res Rev. September-December 1992; 17(3):187-214, incorporated by reference herein in its entirety.
27. Deutsch J, Rapoport S I, Purdon A D. Relation between free fatty acid and acyl-CoA concentrations in rat brain following decapitation. Neurochem Res. July 1997; 22(7):759-765, incorporated by reference herein in its entirety.
28. Folch J, Lees M, Sloane Stanley G H. A simple method for the isolation and purification of total lipides from animal tissues. J Biol. Chem. May 1957; 226(1):497-509, incorporated by reference herein in its entirety.
29. Skipski V P, Good J J, Barclay M, Reggio R B. Quantitative analysis of simple lipid classes by thin-layer chromatography. Biochim Biophys Acta. Jan. 10 1968; 152(1):10-19, incorporated by reference herein in its entirety.
30. Skipski V P, Barclay M, Reichman E S, Good J J. Separation of acidic phospholipids by one-dimensional thin-layer chromatography. Biochim Biophys Acta. Feb. 14 1967; 137(1):80-89, incorporated by reference herein in its entirety.
31. Corey E J, Niwa H, Falck J R. Selective Epoxidation of Eicosa-cis-5,8,11,14-tetraenoic (Arachidonic) Acid and Eicosa-cis-8,11,14-trienoic Acid. J Am Chem Soc 1979; 101:1586-1587, incorporated by reference herein in its entirety.
32. Seltzman H H, Fleming D N, Thomas B F, et al. Synthesis and pharmacological comparison of dimethylheptyl and pentyl analogs of anandamide. J Med. Chem. Oct. 24 1997; 40(22):3626-3634, incorporated by reference herein in its entirety.
33. Miyaura N, Ishiyama T, Sasaki H, Ishikawa M, Satoh M, Suzuki A. Palladium-Catalyzed Inter- and Intramolecular Cross-Coupling Reactions of B-Alkyl-9-borabicyclo[3.3.1]nonane Derivatives with 1-Halo-1-alkene or Haloarenes. Synthesis of Functionalized Alkenes, Arenes, and Cycloalkenes via a Hydroboration-Coupling Sequence. J Am Chem Soc 1989; 111:314-321, incorporated by reference herein in its entirety.
34. Gopal V R, Jagadeesh S G, Reddy Y K, Bandyopadhyay A, Capdevila J H, Falck J R. A practical, stereospecific route to 18-, 19-, and 20-hydroxyeicosa-5(Z),8(Z),11(Z),14(Z)-tetraenoic acids (18-,19-, and 20-HETEs). Tetrahedron Letters 2004; 45:2563-2565, incorporated by reference herein in its entirety.
35. Coenen H H, Elsinga P H, Iwata R et al. Fluorine-18 radiopharmaceuticals beyond [¹⁸F]FDG for use in oncology and neurosciences. Nucl Med Biol 2010 October; 37(7):727-40, incorporated by reference herein in its entirety.
36. Rapoport S I. Arachidonic acid and the brain. J Nutr. December 2008; 138(12):2515-2520, incorporated by reference herein in its entirety.
37. Sun G Y, MacQuarrie R A. Deacylation-reacylation of arachidonoyl groups in cerebral phospholipids. Annals of the New York Academy of Sciences. 1989; 559:37-55, incorporated by reference herein in its entirety.
38. Shimshoni J A, Basselin M, Li L O, Coleman R A, Rapoport S I, Modi H R. Valproate uncompetitively inhibits arachidonic acid acylation by rat acyl-CoA synthetase 4: relevance to valproate's efficacy against bipolar disorder. Biochimica et biophysica acta. March 2011; 1811(3):163-169, incorporated by reference herein in its entirety.
39. Shindou H, Hishikawa D, Harayama T, Yuki K, Shimizu T. Recent progress on acyl CoA: lysophospholipid acyltransferase research. Journal of lipid research. April 2009; 50 Suppl:S46-51, incorporated by reference herein in its entirety.
40. Puntoni M, Marra D, Zanardi S, Decensi A. Inflammation and cancer prevention. Ann Oncol. September 2008; 19 Suppl 7:vii225-229, incorporated by reference herein in its entirety.
41. Vasto S, Carruba G, Candore G, Italiano E, Di Bona D, Caruso C. Inflammation and prostate cancer. Future Oncol. October 2008; 4(5):637-645, incorporated by reference herein in its entirety.
42. Pichika R, Taha A, Gao F, Kotta K, Cheon Y, Chang L, Kiesewetter D, Rapoport S, Eckelman W. The Synthesis and In Vivo Pharmacokinetics of Fluorinated Arachidonic Acid: Implications for Imaging Neuroinflammation. J Nucl Med. September 2012; 53(9):1383-1391, incorporated by reference herein in its entirety.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Also, everywhere “comprising” (or its equivalent) is recited, the “comprising” is considered to incorporate “consisting essentially of” and “consisting of.” Thus, an embodiment “comprising” (an) element(s) supports embodiments “consisting essentially of” and “consisting of” the recited element(s). Everywhere “consisting essentially of” is recited is considered to incorporate “consisting of.” Thus, an embodiment “consisting essentially of” (an) element(s) supports embodiments “consisting of” the recited element(s). Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of diagnosis of the extent of neuroinflammation in a subject, said method comprising:
administering an ¹⁸F-labeled arachidonic acid to a subject in need thereof;
obtaining a positron emission tomography (PET) scan of said subject; and
determining the extent of neuroinflammation of said subject from said PET scan.

2. The method of claim 1, wherein said ¹⁸F-labeled arachidonic acid is [20-¹⁸F] arachidonic acid.

3. The method of claim 1, wherein said neuroinflammation is associated with Alzheimer's disease, bipolar disorder, schizophrenia, Parkinson's disease, HIV-1 associated dementia, brain trauma, epilepsy, cancer or bacterial infections.

4. The method of claim 2, wherein said neuroinflammation is associated with Alzheimer's disease, bipolar disorder, schizophrenia, Parkinson's disease, HIV-1 associated dementia, brain trauma, epilepsy, cancer or bacterial infections.

5. A method for producing methyl 20-(p-toluenesulfonyloxy)-5,8,11,14-eicosatetraenoate, said method comprising reacting methyl 20-hydroxy-5,8,11,14-eicosatetraenoate with tosyl chloride and triethylamine in dichloromethane.

6. A method for producing methyl 20-fluoroarachidonate, said method comprising reacting methyl 20-(p-toluenesulfonyloxy)-5,8,11,14-eicosatetraenoate with tetra-n-butylammonium fluoride in tethydrofuran.

7. A method for separation of geometric isomers of methyl 20-hydroxy-5,8,11,14-eicosatetraenoate, said method comprising subjecting a mixture of E and Z isomers of methyl 20-hydroxy-5,8,11,14-eicosatetraenoate to flash column chromatography.
